(19) United States
(12) Patent Application Publication
(10) Pub. No.: US 2009/0080596 A1
(43) Pub. Date: Mar. 26, 2009

(54) RADIOGRAPHIC EQUIPMENT

(75) Inventors: Brian David Sowerby, New South Wales (AU); James Richard Tickner, South Australia (AU); Gregory John Roach, New South Wales (AU)

Correspondence Address: SUGHRUE MION, PLLC, 2100 PENNSYLVANIA AVENUE, N.W., SUITE 800, WASHINGTON, DC, 20037, US

(73) Assignee: COMMONWEALTH SCIENTIFIC AND INDUSTRIAL RESEARCH OR, Australian Capital Territory (AU)

(21) Appl. No.: 11/664,614

(22) PCT Filed: Oct. 5, 2005
(86) PCT No.: PCT/AU2005/001522

§ 371 Date: Aug. 29, 2008

(30) Foreign Application Priority Data
Oct. 5, 2004 (AU) 2004905744

(51) Int. Cl. A61B 6/00

(52) U.S. Cl. 378/5

(57) ABSTRACT

The invention concerns radiographic equipment for forming an image of an interior of an object. The equipment comprises a source of X-ray or gamma-ray radiation having two or more energies and operable to irradiate an object to be scanned and a radiation source producing neutrons operable to irradiate the object. The equipment also comprises a radiation detector array having a plurality of pixels, each sensitive to and arranged with respect to the X-ray or gamma-ray radiation source and the neutron producing radiation source and operable to measure the intensity of each type of radiation transmitted through the object; means to process the intensity of each type of radiation, to determine the attenuation of each type of radiation having passed through the object, and to form an image indicative of the shape and composition of the object's interior.

### TECHNICAL FIELD

This invention concerns radiographic equipment and a method for forming an image of an interior of an object. In particular the invention concerns radiographic equipment for the detection of concealed articles, substances and materials in items such as aircraft luggage, packages, and similar items.

### BACKGROUND ART

X-ray radiography, where the attenuation of X-rays is measured between a source placed on one side of the object to be examined and a screen or detector on the opposite side, was first demonstrated by Rõntgen in 1895. Images can be readily obtained showing the size and shape of objects inside a suitcase or package. X-ray images can easily be obtained with excellent spatial resolution, showing fine details of objects being scanned. However, the composition of these objects cannot be determined using a single X-ray energy.

A significant and well-known enhancement comprises obtaining two separate X-ray transmission images at different X-ray energies [1], a method which has been applied to security imaging [2]. The attenuation of high energy X-rays depends primarily of the mass of material between the source and detector. The attenuation of lower energy X-rays depends on both the mass and composition of the material, with higher atomic number materials absorbing the X-rays more strongly. Consequently, the two X-ray images can be processed to show both the shapes and average atomic number of objects being imaged.

The main deficit of the so-called dual energy X-ray image technique is that whilst it offers excellent discrimination between organic and inorganic materials, it offers little or no ability to distinguish between different classes of organic substances. In particular, it is difficult to use the method to separate benign organic materials such as plastics, clothing or foodstuffs from items such as illicit drugs or explosives. Although these materials have different densities, density cannot be inferred from an X-ray image unless additional information on an object's thickness is available.

In contrast, the attenuation of neutrons varies widely between different materials, both organic and inorganic, and varies strongly as a function of neutron energy. The principle of measuring neutron transmission at multiple energies to improve material identification is well known [3]. However, practical application of this technique for scanning items such as luggage is limited. The brightness of readily available neutron sources is relatively low compared to X-ray sources and neutron detectors typically have low spatial resolution and detection efficiency compared to X-ray detectors. Neutron detectors providing energy discrimination are complicated, relying on either nanosecond time-of-flight measurements or spectral unfolding techniques to infer the incident neutron energy spectrum. Consequently, neutron radiography systems are typically too slow, have too poor a spatial resolution and insufficient material discrimination to form the basis of practical luggage or parcel scanners.

### DISCLOSURE OF THE INVENTION

In a first aspect, the invention is radiographic equipment for forming an image of an interior of an object, the equipment comprising:

a source of X-rays or gamma-rays, the source having two or more energies and operable to irradiate an object to be scanned;

a radiation source producing neutrons operable to irradiate the object;

a radiation detector array comprised of a plurality of pixels, each sensitive to and arranged with respect to the X-ray or gamma-ray radiation source and the neutron producing radiation source to measure the intensity of each type of radiation transmitted through the object; and

processing means to process an intensity of each type of radiation, to determine the attenuation of the radiation as it passes through the object, and to form an image indicative of the shape and composition of the object's interior.

The object may be a suitcase, luggage, a package, or other like item.

The X-ray radiation source may comprise an X-ray tube operable to produce X-rays with a wide range of energies up to the maximum electron energy (typically about 150 keV for luggage scanners and 450 keV for pallet scanners).

The gamma-ray radiation source may comprise one or more radio-isotopes producing high and low energy X-rays or gamma-rays. The radiation source may be contained in a shield made of a material such as lead which is substantially opaque to X-rays and gamma-rays. A slot cut into the shield may serve to define fan-shaped radiation beams, incident on the detector array and though which the object to be scanned is passed.

The neutron producing radiation source may be a sealed tube neutron source, producing neutrons via the deuterium-tritium (DT) or deuterium-deuterium (DD) fusion reactions. Optionally, the neutron source may be a radio-isotope source such as, but not limited to ²⁵²Cf or ²⁴¹Am—Be. Furthermore neutrons may be produced using a particle accelerator using reactions such as D(d,n)³He, ⁷Li(p,n)⁷Be or ⁹Be(d,n)¹⁰B.

The neutron source may be contained in a shield made from a material substantially opaque to neutrons, such as polyethylene, concrete, wax or iron. The shield may also contain a thermal neutron absorbing material such as compounds of boron or lithium. A slot cut into the shield may serve to define a fan-shaped radiation beam directed at the neutron radiation detector array.

The radiation detector array may include an X-ray or gamma-ray radiation detector array and a separate neutron radiation detector array.

The X-ray or gamma-ray radiation detector array may be single detector array which is capable of distinguishing the energies of incident X-rays. Such a detector may be used to measure the transmission of high- and low-energy X-rays. Optionally, two separate detector arrays may be used, with the arrays designed to respond preferentially to high or low energy X-rays. Non limiting examples of these energies are 150 keV and 60 keV respectively.

The dual-energy X-ray/gamma-ray source and X-ray or gamma-ray radiation detector array may be an existing dual-energy X-ray scanner.

The neutron radiation detector array may comprise an array of plastic scintillator pixels, read out using photomultiplier tubes or photodiodes. The scintillator material may be selected such that its emission wavelength is substantially matched to the response of the photodiodes. Optionally, the neutron radiation detector array may comprise an array of cells filled with a liquid organic scintillator read out using photomultipliers or photodiodes. Advantageously, the timing properties of the light signals produced by neutrons and X-rays or gamma-rays in a liquid scintillator differ, allowing X-ray or gamma-ray backgrounds in the neutron detectors to be reduced. This technique is commonly referred to as pulse shape discrimination (PSD). Optionally, the neutron radiation detector array may comprise an array of plastic scintillators, read out using wavelength shifting fibres. Optionally, the array may take the form of one or more bubble chambers, detecting neutrons via the production of bubbles in a super-critical liquid. The bubble chamber detector may be read out either by optical imaging or piezo-electric detection of the bubbles. Other alternative neutron detectors include, but are not limited to: stilbene crystals with PSD, compressed gas counters (such as xenon), a neutron sensitive scintillation screen with a CCD camera, and microchannel plate detectors (with amorphous silicon readout).

The X-ray and gamma-ray radiation source and detector and the neutron producing radiation source and detector may be similarly configured, such that rays passing from either source to the respective detector have the same, or substantially the same path through the object being scanned, possibly displaced if separate arrays are used. In particular, the distance between the radiation sources and their respective detector arrays may be the same, or substantially the same, and the arrays have the same, or substantially the same length. This facilitates registration of the X-ray and neutron images.

The equipment may further comprise transport means for transporting the object through an X-ray or gamma-ray beam produced by the X-ray or gamma-ray radiation source and a neutron beam produced by the neutron producing radiation source. Optionally the object to be scanned may be stationary and the transport means may be arranged such that the respective radiation sources and detector array are moved in synchronicity on either side of the object.

Rotation means may be provided such that the radiation sources and the detector array are rotatable relative to the object to be scanned.

The processing means may be operable, from the attenuation determinations, to compute mass attenuation coefficient images for each pixel, with pixel values mapped to different colours. The processing means may be further operable to obtain a cross section ratio image between a pair of mass attenuation coefficient images. Automatic material identification based on the measured cross sections may be performed. Moreover, the proportions in which the cross section ratio images are combined may be operator adjustable.

The transport means or translating means for translating the object through the scanner may comprise a conveyor belt or similar means on which the object to be scanned is placed. Optionally, the object may be held fixed and the radiation sources and detector moved in tandem past the object. Multiple views may be obtained by rotating the object relative to the sources and the detector array or by using multiple sources and detector arrays.

The processing means for producing and displaying images of scanned objects may comprise a computer or similar system. The processing means may include an attenuation measurement means which may store the measurements into a 2-dimensional array. The computer or similar system may operate to read out the X-ray or gamma-ray and neutron detector arrays at regular intervals. The time between readouts may be selected such that during this interval the object being scanned travels a distance similar to the distance between neighbouring pixels of the array. In this way, a 2-dimensional image of the radiation flux may be obtained. This flux image may be conveniently converted to a transmission image by dividing the flux at each detector pixel by the flux obtained at the same pixel when either no intervening object is present or when fewer objects are present.

The attenuation measured for the higher energy X-ray or gamma-ray radiation is most nearly proportional to the mass material in the radiation beam and may be used to determine the brightness of the pixel. Appropriate combinations of the high and low energy X-ray or gamma-ray beams and of the high energy X-ray and neutron beams can be used to estimate the material composition. This information may be used to select the colour or hue of the pixel. The operator of the scanner may be provided with controls to enable them to manipulate the brightness, contrast and colour of the image display to facilitate the identification of suspect items and materials.

The equipment may further comprise a display device for displaying the image to an operator. It will be appreciated that the display device may be a colour monitor, LCD display screen, plasma flat panel, or the like.

In a second aspect, the invention is a method for forming an image of an interior of an object, the method comprising:

generating a beam of X-ray or gamma ray radiation and a beam of neutron radiation, where the beam of X-ray or gamma ray radiation has two or more energies;

positioning an object in the path of the beam of X-ray or gamma ray radiation and the beam of neutron radiation;

measuring, within a plurality of pixels, an intensity of X-ray or gamma-ray radiation and neutron radiation transmitted through the object;

determining an attenuation of the X-ray or gamma-ray and neutron radiation; and

further processing both types of radiation measurements to form an image indicative of the shape and composition of the object's interior.

The method may comprise collimating the beam of X-ray or gamma ray radiation and the beam of neutron radiation such that respective fan shaped radiation beams are incident on the plurality of pixels.

The method may comprise filtering the measure of neutron radiation to reduce the presence of gamma-ray background radiation.

The method may comprise computing mass attenuation coefficient images for each pixel, with pixel values mapped to different colours. The method may further comprise computing a cross section ratio image between a pair of mass attenuation coefficient images.

The method may further comprise automatically identifying the objects composition based on the measured cross sections.

Processing both types of radiation measurements to form an image may include the step of combining images obtained by determining the attenuation of transmitted X-rays or gamma rays with two or more energies with an image obtained by determining the attenuation of neutrons.

An advantage of at least one embodiment of the invention is that the dual energy X-ray/gamma-ray technique offers excellent discrimination between organic and inorganic materials whilst the addition of neutron transmission information to an image allows much better separation of material compositions. This facilitates the interpretation of images of scanned objects and significantly improves the detection rate for illicit materials such as explosives.

The dual-energy X-ray/gamma-ray system furnishes high-resolution images with good discrimination between inorganic and organic materials. The addition of a neutron image, based on a measurement (integrated over neutron energy) of the transmission of neutrons from a source to a detector array, provides improved material separation, particularly between different classes of organic substances. The neutron image can have considerably lower spatial resolution than the X-ray image as it is only used to provide composition information, with the high-resolution shape and detail information coming mainly from the X-ray image. The extra composition information facilitates the interpretation of images of scanned objects and improves the detection rate for illicit or contraband materials.

### BRIEF DESCRIPTION OF DRAWINGS

An example of the invention will now be described with reference to the accompanying drawings in which:

FIG. 1 is a schematic illustration of radiographic equipment for forming an image of an interior of an object;

FIG. 2 is a bar graph which plots the cross-section ratios for high (150 keV) and low (60 keV) energy X-rays for a variety of materials;

FIG. 3 is a bar graph which plots the cross-section ratios for 2.5 MeV neutrons and high (150 keV) energy and X-rays for a variety of materials;

FIG. 4 is a graph which plots the cross-sections ratios for high (150 keV) and low (60 keV) energy X-rays against the cross-section ratios for 2.5 MeV neutrons and high (150 keV) energy X-rays for a variety of materials;

FIG. 5a is an image of a simulated suitcase containing a variety of benign and contraband materials obtained from a conventional dual-energy X-ray scanner; and

FIG. 5b is an image of the simulated suitcase obtained when using radiographic equipment as illustrated in FIG. 1.

### BEST MODE FOR CARRYING OUT THE INVENTION

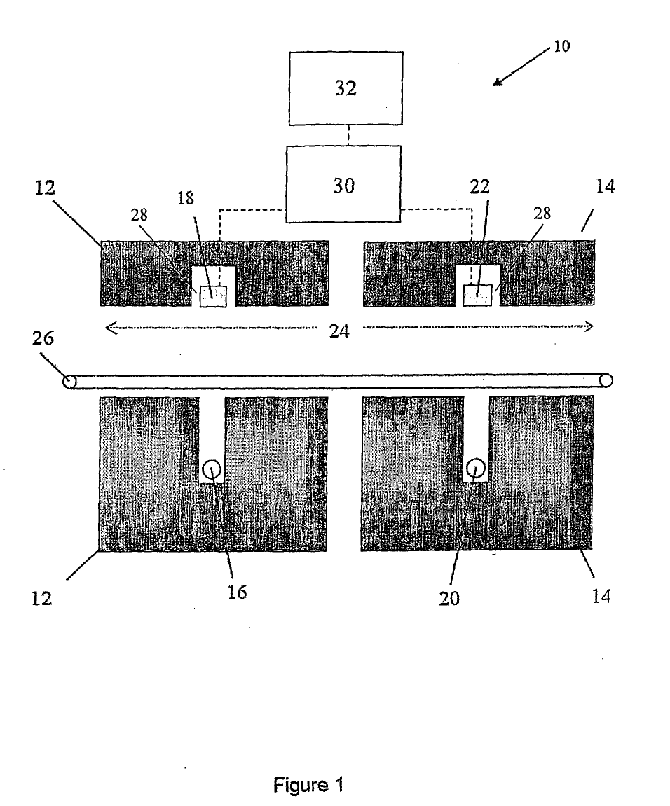

FIG. 1 illustrates radiographic equipment 10 for forming an image of an object in the form of a suitcase (not shown). The equipment 10 includes a pair of shielding blocks 12 and 14. Shielding block 12 contains an X-ray tube source 16 capable of generating dual energy X-rays and a dual-energy X-ray detector 18. Shielding block 14 contains a ²⁵²Cf neutron source 20 and a neutron detector 22. A tunnel 24 passes through shielding blocks 12 and 14 and a conveyor belt 26, passing through tunnel 24, is used to transport the suitcase and other like objects through the equipment 10.

Slots 28 in shielding blocks 12 and 14 define fan-shaped beams of X-ray and neutron radiation that are incident on detectors 18 and 22 respectively. Advantageously, in addition to defining the radiation beams, shielding blocks 12 and 14 also provide radiological shielding, protecting operators of the equipment 10 from exposure to radiation.

The X-ray tube 16 is operated at a high voltage such that it produce X-rays with maximum energies within the range of 150-450 keV. The dual-energy X-ray detector 18 includes arrays of CsI(T1) crystals optically coupled to arrays of photodiodes. The arrays are operated in current mode, in which case two arrays are required with appropriate filtering to discriminate between high and low energy X-rays, where the low and high energy X-rays are in the range 60 keV to 450 keV respectively. The pixel size of the X-ray detectors is around 1 mm, or as small as can be practically obtained.

The neutron source 20 comprises a ²⁵²Cf radioisotope source producing approximately 10⁸ neutrons per second. A filter (not shown) is positioned between the radiation source 20 and the neutron detector 22 to attenuate gamma-ray radiation incidentally produced by the source 20.

The neutron detector 22 comprises cells filled with an organic liquid scintillator with pulse-shape discrimination properties, such as NE-213 or BC-501A. The cells, which are optically isolated, are coupled to multiple photomultipliers through a transparent medium. The thickness of the medium is chosen to allow the light from each cell to spread out and reach multiple photomultipliers. Measurement of the light division between neighbouring photomultipliers allows the cell in which the radiation was incident to be deduced. The total light detected provides a measure of the energy of the incident radiation and the timing distribution of the light pulse allows neutrons and gamma-rays to be discriminated.

The neutron cells, or pixels, have transverse dimensions of approximately 10×10 mm. The pixels are made long enough to render them substantially opaque to the neutron radiation, increasing the detection efficiency of the system.

Outputs from the detectors 18 and 22 are processed by a processor 30 to form an image indicative of the shape and composition of an interior of the object. The image is displayed on a computer display device 32 which is not necessarily in proximity to the processor 30.

The conveyor belt 26 is operated at a speed in the range of one to ten metres per minute, allowing approximately one to ten objects of suitcase size to be scanned per minute. The processor 30 reads out the X-ray and neutron detector arrays at regular intervals. The X-ray detectors 18 are read out and reset every time the object has moved through a distance equal to the X-ray detector array pixel size, nominally one mm. Similarly, the cells of the neutron detector 22 are readout and reset every time the suitcase has moved through a distance equal to the neutron detector array pixel size, nominally ten mm. This results in three images; two high resolution X-ray images and one lower resolution neutron image.

Suppose that the high and low energy X-ray fluxes for a particular pixel are I_{H} and I_{L} respectively. Let the fluxes obtained when no object is present be I⁰_{H} and I⁰_{L} respectively. The mass of material in X-ray beam, m, and the cross-section ratio R₁ can then be estimated from the relations:

m=−k log(I_{H}/I⁰_{H}) (1)

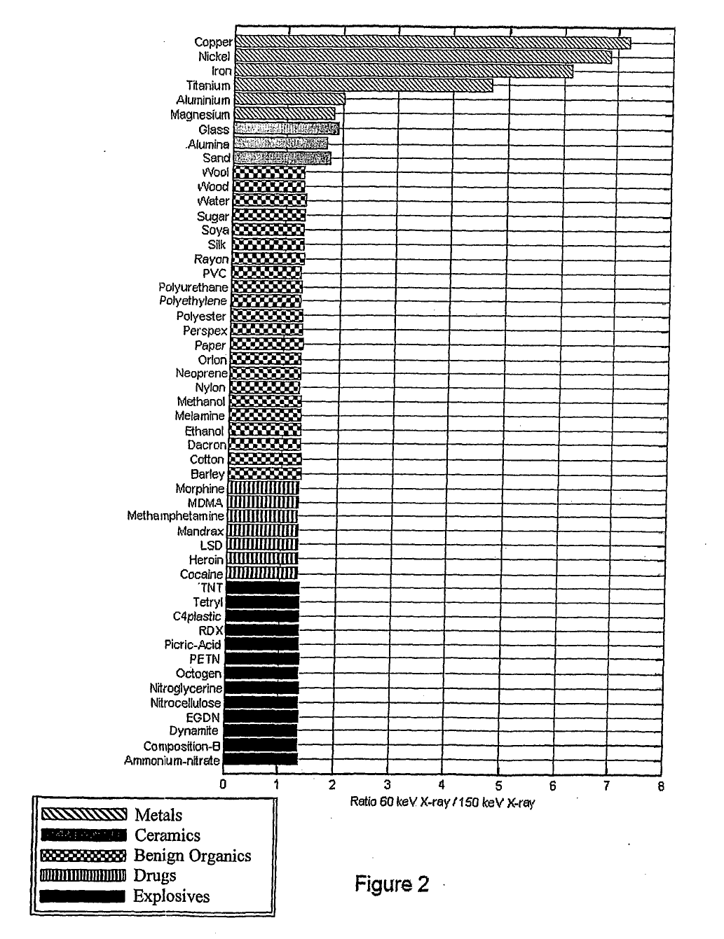

R₁=log(I_{L}/I⁰_{L})/log(I_{H}/I⁰_{H}) (2)

where k is a constant parameter than depends on the energy of the high-energy X-rays. The discrimination between inorganic and organic materials resulting from the measurement of R₁ is illustrated in FIG. 2. The lower and higher X-ray energies are 60 and 150 keV respectively. In practice, an X-ray tube source produces X-rays with a continuous range of energies and relations (1) and (2) need to be replaced with appropriate integrations over the X-ray source energy spectrum.

Due to the lower resolution of the neutron image, a single neutron image pixel will correspond to multiple X-ray image pixels. The neutron/X-ray cross-section ratio R₂ can be estimated from the relation:

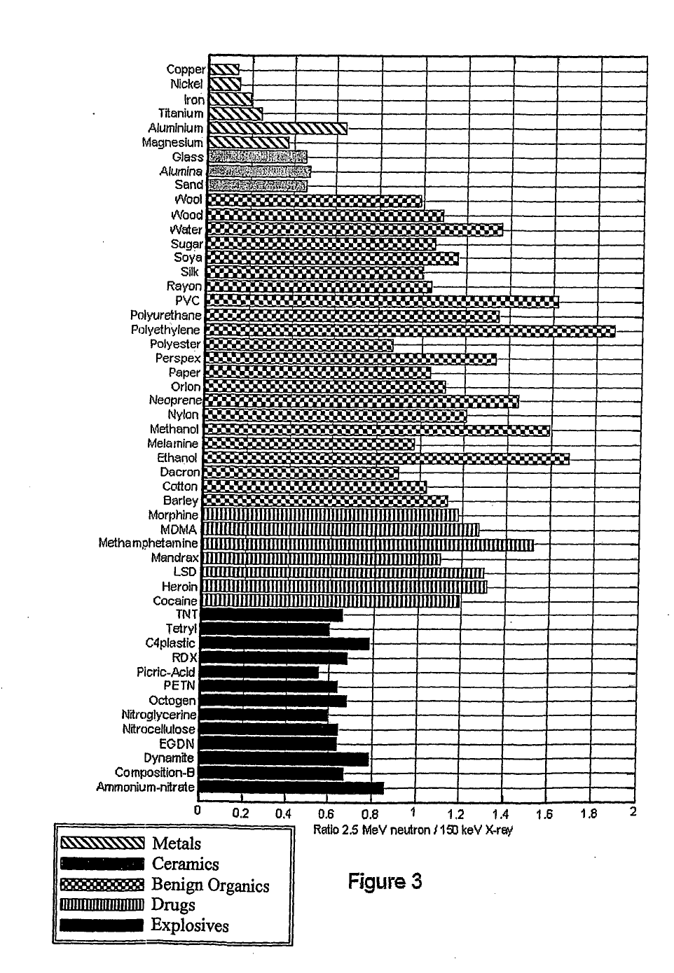

R₂=log(I_{N}/I⁰_{N})/average[log(I_{H}/I⁰_{H})] (3)

where the average [ ] extends over all of the X-ray image pixels corresponding to a particular neutron pixel. Here, I_{N} is the measured neutron flux and I⁰_{N} is the neutron flux obtained when no object is present. The extra discrimination between organic materials provided by a measurement of R₂ is illustrated in FIG. 3.

The parameters m, R₁ and R₂ are used to determine the colour of each pixel in the image that is presented to the operator of the apparatus 10. The parameter m is used to determine the brightness of the pixel. Pixels with m close to zero (little or no material in beam) could for example be coloured white. As m increases in value, pixels are coloured increasingly strongly, with the colour determined by the values of R₁ and R₂.

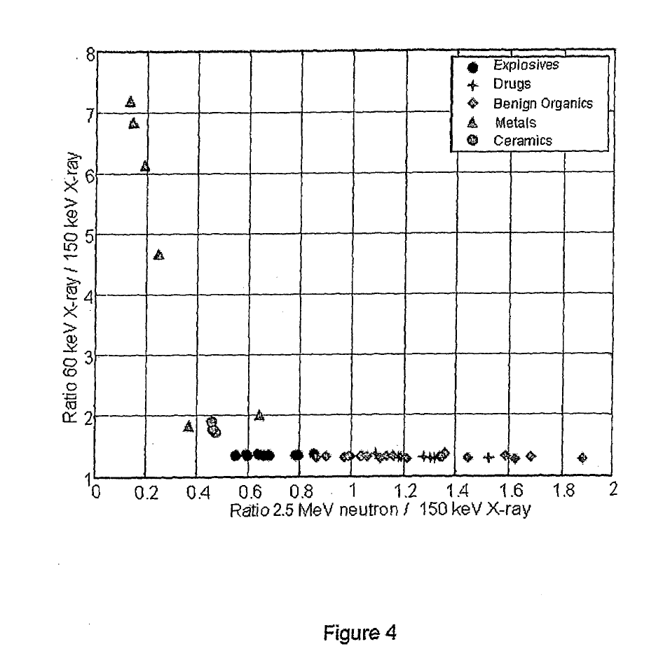

FIG. 4 plots the cross-section ratio R₂ against the cross-section ratio R₁ for a variety of benign materials, explosives and narcotics.

In a conventional dual energy X-ray scanner, pixels with high R₁ values, which correspond to material with a high atomic number such as metals, are coloured blue. Intermediate R₁ valued materials are coloured green, and the lowest R₁ valued materials (typically organic substances) are coloured orange or brown. With the additional information present in the R₂ parameter this colour scheme can be extended. Ideally, materials with small R₁ values would be mapped into warm colours (purple, red, orange, and yellow) according to their R₂ values. Existing scanner operators would be familiar with the basic image presentation, but the separation of different classes of organics would greatly simplify the problem of identifying threat materials.

This scheme is particularly powerful at identifying concealed explosives. Due to their relatively low hydrogen content, most explosives have substantially different R₂ values from benign organic materials, whilst having R₁ values that separate them from inorganic materials. This is especially the case when a source emitting lower energy neutrons is used, such as a DD sealed tube neutron generator or a ²⁵²Cf fission radioisotope, as shown by the cross-section ratios plotted in FIG. 3.

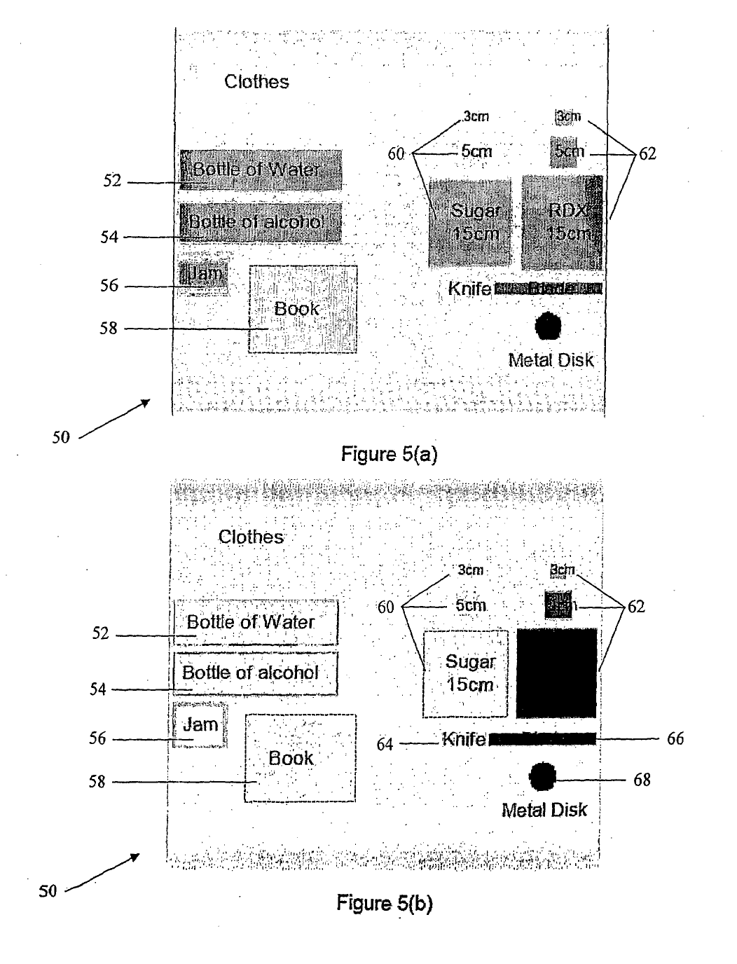

FIGS. 5(a) and 5(b) show simulated images of a suitcase 50 containing both benign materials and concealed explosives. The suitcase 50 measures 80×60×20 cm and contains bottles of water 52 and alcohol 54, a jar of jam 56, a three cm thick book 58, three packages each of sugar 60 and RDX explosive 62 having sizes of fifteen cm, five cm and three cm, a knife handle 64 having a metal blade 66 and a metal disc 68. The remainder of the suitcase is filled with clothing 70. FIG. 5(a) shows the image that would be obtained using an existing dual-energy X-ray scanner. FIG. 5(b) illustrates the advantages that neutron radiography adds to the conventional dual energy X-ray technique. Different classes of organic material can be readily distinguished, with even the smallest quantity of explosive 62 clearly showing.

Whilst the X-ray tube 16 illustrated in FIG. 1 has been described as being operated at a high voltage such that it produce X-rays with energies within the range 150-450 keV, a radioisotope source such as ¹³³Ba, which produces gamma-rays having energies of around 80 keV and 350 keV may be used. A combination of radioisotopes such as ²⁴¹Am (producing 60 keV gamma-rays) and ¹³⁷Cs (producing 662 keV gamma-rays) may also be used.

In the embodiment described above, the dual-energy X-ray detector 18 includes arrays of CsI(T1) crystals optically coupled to arrays of photodiodes. The arrays may be operated in pulse mode where individual X-rays and detected, sorted according to energy and counted.

In an optional embodiment, the neutron source 20 comprises a sealed tube DD neutron generator, producing approximately 10⁸ neutrons per second, or as high an output as can be practically obtained. In a still optional embodiment, the neutron source 20 consists of a sealed tube DT neutron generator, producing approximately 10⁸ neutrons per second. In a still optional embodiment the neutron source 20 consists of an alpha-beryllium radioisotope source such as ²⁴¹Am—Be, producing approximately 10⁸ neutrons per second, or as high an output as can be practically obtained.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

[1] Alvarez R. E. and Macovski A. (1976), Energy-selective Reconstructions in X-ray Computerized Tomography, Phys. Med. Biol. 21(5) p 733.
[2] Stein J. A., Krug K. D. and Taylor A. L. (1992) Baggage inspection method with dual energy X-ray discrimination—using exposure to dual energise allows processing of comparative attenuation data to identify presence of material esp. explosives, WO9202892.
[3] Miller T. G. (1995) Apparatus for radiographic/tomographic detection—uses white neutron beam to measure attenuation of non-scattered neutrons and compare it with known neutron cross-sections.

1-39. (canceled)

40. Radiographic equipment for forming an image of an interior of an object, the equipment comprising:
a source of X-rays, or gamma-rays, operable to irradiate an object to be scanned, the source operable to emit at least two different energies of radiation;
a radiation source producing neutrons operable to irradiate the object;
a radiation detector array comprised of a plurality of pixels, each sensitive to and arranged with respect to the X-ray or gamma-ray radiation source and the neutron producing radiation source the detector array providing data corresponding to the intensity of transmitted X-ray or gamma-ray radiation at each of the different energies and the neutron radiation through the object; and
a processor to process the intensity of the transmitted X-ray or gamma-ray radiation of the at least two different energies and the intensity of transmitted neutron radiation, to determine the attenuation of the radiation as it passes through the object, and to form an image indicative of the shape and composition of the object's interior.

41. Radiographic equipment according to claim 40, where the X-ray source comprises an X-ray tube operable to produce X-rays with maximum energies within the range of 150 to 450 keV.

42. Radiographic equipment according to claim 40, where the gamma-ray radiation source comprises at least one radioisotope producing high and low energy X-rays or high and low energy gamma-rays, with energies within the range of 60 keV to 662 keV.

43. Radiographic equipment according to claim 40, where the X-ray or gamma-ray radiation source is substantially surrounded by a shield which is substantially opaque to X-rays and gamma-rays.

44. Radiographic equipment according to claim 43, where a slot is cut into the shield which serves to define a fan-shaped radiation beam emitted from the source such that the fan shaped beam is incident on the detector array.

45. Radiographic equipment according to claim 40, where the neutron producing radiation source is a sealed tube neutron source, operable to produce neutrons via a deuterium-tritium (DT) fusion reaction.

46. Radiographic equipment according to claim 40, where the neutron producing radiation source is a sealed tube neutron source, operable to produce neutrons via a deuterium-deuterium (DD) fusion reaction.

47. Radiographic equipment according to claim 40, where the neutron producing radiation source is substantially surrounded by a neutron shield which is substantially opaque to neutrons.

48. Radiographic equipment according to claim 47, where a slot is cut into the shield which serves to define a fan-shaped radiation beam emitted from the neutron producing radiation source such that the fan shaped beam is incident on the detector array.

49. Radiographic equipment according to claim 40, where the radiation detector array includes an X-ray or gamma-ray radiation detector array and a separate neutron radiation detector array.

50. Radiographic equipment according to claim 49, where the X-ray or gamma-ray radiation detector array is a single detector array which is capable of distinguishing the energies of incident X-rays.

51. Radiographic equipment according to claim 49, where the X-ray or gamma-ray radiation detector array comprises two separate detector arrays, with the first array is configured to respond preferentially to high energy X-rays and the second array configured to respond preferentially to low energy X-rays.

52. Radiographic equipment according to claim 49, where the neutron radiation detector array comprises an array of plastic scintillators coupled to one or more photodetectors.

53. Radiographic equipment according to claim 52, where the photodetectors are photomultiplier tubes.

54. Radiographic equipment according to claim 52, where the photodetectors are photodiodes and where the scintillator material is selectable to have an emission wavelength substantially matched to the response of the photodiodes.

55. Radiographic equipment according to claim 40, where a rotation device is provided such that the radiation sources and the detector array are rotatable relative to the object to be scanned.

56. Radiographic equipment according to claim 40, where the processor is operable, from the attenuation determinations, to compute mass-attenuation coefficient images for each pixel.

57. Radiographic equipment according to claim 56, where the processor is operable to produce coloured images, with the colours being determined from cross-section ratios, formed between pairs of mass-attenuation coefficients.

58. Radiographic equipment according to claim 57, where the processor is operable to perform automatic material identification based on the computed cross-section ratios.

59. A method for forming an image of an object's interior, the method comprising:
generating a beam of X-ray or gamma ray radiation at two or more different energies and a beam of neutron radiation;
translating an object through the path of the beam of X-ray or gamma ray radiation and the beam of neutron radiation;
measuring, within a plurality of pixels, an intensity of X-ray or gamma-ray radiation at each of the two or more energies and an intensity of the neutron radiation, transmitted through the object;
determining an attenuation of the X-ray or gamma-ray radiation at each of the two or more energies and the neutron radiation; and
further processing both types of the attenuation measurements to form an image indicative of the shape and composition of the object's interior.

60. A method for forming an image of an object's interior according to claim 59, further comprising collimating the beam of X-ray or gamma ray radiation and the beam of neutron radiation such that respective fan shaped radiation beams are incident on the plurality of pixels.

61. A method for forming an image of an object's interior according to claim 59, further comprising computing mass attenuation coefficient images for each pixel.

62. A method for forming an image of an object's interior according to claim 61, further comprising computing pixel colours based on cross-section ratios formed between pairs of mass attenuation coefficient images.

63. A method for forming an image of an object's interior according to claim 62, further comprising automatically identifying the object's composition based on the computed cross-section ratios.

64. A method for forming an image of an object's interior according to claim 59, comprising further processing both types of the attenuation measurements to perform automatic identification of threat materials, in particular explosive materials.
